(19) United States
(12) Patent Application Publication
(10) Pub. No.: US 2010/0168603 A1
(43) Pub. Date: Jul. 1, 2010

(54) BRAIN STATE ANALYSIS BASED ON SELECT SEIZURE ONSET CHARACTERISTICS AND CLINICAL MANIFESTATIONS

(76) Inventors: David M. HIMES, Seattle, WA (US); Sara M. Rolfe, Seattle, WA (US); David E. Snyder, Bainbridge Island, WA (US)

Correspondence Address: NEUROVISTA / SHAY GLENN, 2755 CAMPUS DRIVE, SUITE 210, SAN MATEO, CA, 94403, US

(21) Appl. No.: 12/646,783

(22) Filed: Dec. 23, 2009

Related U.S. Application Data
(60) Provisional application No. 61/140,592, filed on Dec. 23, 2008.

(51) Int. Cl. A61B 5/0476

(52) U.S. Cl. 600/544

(57) ABSTRACT

Systems and methods for developing a brain state analysis system using subject EEG data are provided. The analysis system distinguishes clinical from subclinical electrographic seizures and optionally distinguishes among different seizure onset characteristics. An algorithm trained on only clinical electrographic seizures may predict clinical seizures more accurately with fewer perceived false positives. In addition, algorithms trained on a particular onset condition may distinguish and advise on that onset condition when used by the patient.

### CROSS-REFERENCE TO RELATED APPLICATIONS

This application claims the benefit of U.S. Provisional Application No. 61/140,592, filed Dec. 23, 2008, which is incorporated herein by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### BACKGROUND OF THE INVENTION

The present invention relates generally to systems and methods, for sampling and processing one or more physiological signals from a subject. More specifically, the present invention relates to monitoring of one or more neurological signals from a subject to determine a subject's susceptibility to a neurological event, communicating the subject's susceptibility to the subject and/or to another monitor, and optionally treating the patient acting to, e.g., reduce severity of seizures and/or prevent seizures.

Epilepsy is a neurological disorder of the brain characterized by chronic, recurring seizures. Seizures are a result of uncontrolled discharges of electrical activity in the brain. A seizure typically manifests itself as sudden, involuntary, disruptive, and often destructive sensory, motor, and cognitive phenomena. Seizures are frequently associated with physical harm to the body (e.g., tongue biting, limb breakage, and burns), a complete loss of consciousness, and incontinence. A typical seizure, for example, might begin as spontaneous shaking of an arm or leg and progress over seconds or minutes to rhythmic movement of the entire body, loss of attention, loss of consciousness, and voiding of urine or stool.

A single seizure most often does not cause significant morbidity or mortality, but severe or recurring seizures (epilepsy) can result in major medical, social, and economic consequences. Epilepsy is most often diagnosed in children and young adults, making the long-term medical and societal burden severe for this population of subjects. People with uncontrolled epilepsy are often significantly limited in their ability to work in many industries and usually cannot legally drive an automobile. An uncommon, but potentially lethal form of seizure is called status epilepticus, in which a seizure continues for more than 30 minutes. This continuous seizure activity may lead to permanent brain damage and can be lethal if untreated.

While the exact cause of epilepsy is often uncertain, epilepsy can result from head trauma (such as from a car accident or a fall), infection (such as meningitis), stroke, or from neoplastic, vascular or developmental abnormalities of the brain. In approximately 70% of epileptic subjects, especially those having forms that are resistant to treatment (i.e., refractory), are idiopathic, or of unknown causes, epilepsy is generally presumed to be an inherited genetic disorder.

Demographic studies have estimated the prevalence of epilepsy at approximately 1% of the population, or approximately 2.5 million individuals in the United States alone. In order to assess possible causes and to guide treatment, epileptologists (both neurologists and neurosurgeons) typically evaluate subjects with seizures with brain wave electrical analysis and imaging studies, such as magnetic resonance imaging (MRI).

While there is no known cure for epilepsy, chronic usage of anticonvulsant and antiepileptic medications can control seizures in most people. For most cases of epilepsy, the disease is chronic and requires chronic medications for treatment. The anticonvulsant and antiepileptic medications do not actually correct the underlying conditions that cause seizures. Instead, the anticonvulsant and antiepileptic medications manage the subject's epilepsy by reducing the frequency of seizures. There are a variety of classes of antiepileptic drugs (AEDs), each acting by a distinct mechanism or set of mechanisms.

AEDs generally suppress neural activity by a variety of mechanisms, including altering the activity of cell membrane ion channels and the susceptibility of action potentials or bursts of action potentials to be generated. These desired therapeutic effects are often accompanied by the undesired side effect of sedation, nausea, dizziness, etc. Some of the fast acting AEDs, such as benzodiazepine, are also primarily used as sedatives. Other medications have significant non-neurological side effects, such as gingival hyperplasia, a cosmetically undesirable overgrowth of the gums, and/or a thickening of the skull, as occurs with phenytoin. Furthermore, some AED are inappropriate for women of child bearing age due to the potential for causing severe birth defects.

An estimated 70% of subjects will respond favorably to their first AED monotherapy and no further medications will be required. However, for the remaining 30% of the subjects, their first AED will fail to fully control their seizures and they will be prescribed a second AED—often in addition to the first—even if the first AED does not stop or change a pattern or frequency of the subject's seizures. For those that fail the second AED, a third AED will be tried, and so on. Subjects who fail to gain control of their seizures through the use of AEDs are commonly referred to as “medically refractory.” This creates a scenario in which 750,000 subjects or more in the United States have uncontrolled epilepsy. These medically refractory subjects account for 80% of the $12.5 billion in indirect and direct costs that are attributable to epilepsy in the United States.

A major challenge for physicians treating epileptic subjects is gaining a clear view of the effect of a medication or incremental medications. Presently, the standard metric for determining efficacy of the medication is for the subject or for the subject's caregiver to keep a diary of seizure activity. However, it is well recognized that such self-reporting is often of poor quality because subjects often do not realize when they have had a seizure, or fail to accurately record seizures.

If a subject is refractory to treatment with chronic usage of medications, surgical treatment options may be considered. If an identifiable seizure focus is found in an accessible region of the brain, which does not involve “eloquent cortex” or other critical regions of the brain, then resection is considered. If no focus is identifiable, there are multiple foci, or the foci are in surgically inaccessible regions or involve eloquent cortex, then surgery is less likely to be successful or may not be indicated. Surgery is effective in more than half of the cases, in which it is indicated, but it is not without risk, and it is irreversible. Because of the inherent surgical risks and the potentially significant neurological sequelae from resective procedures, many subjects or their parents decline this therapeutic modality.

Some non-resective functional procedures, such as corpus callosotomy and subpial transection, sever white matter pathways without removing tissue. The objective of these surgical procedures is to interrupt pathways that mediate spread of seizure activity. These functional disconnection procedures can also be quite invasive and may be less effective than resection.

An alternative treatment for epilepsy that has demonstrated some utility is open loop Vagus Nerve Stimulation (VNS). This is a reversible procedure which introduces an electronic device that employs a pulse generator and an electrode to alter neural activity. The vagus nerve is a major nerve pathway that emanates from the brainstem and passes through the neck to control visceral function in the thorax and abdomen. VNS uses open loop, intermittent stimulation of the left vagus nerve in the neck in an attempt to reduce the frequency and intensity of seizures. See Fisher et al., “Reassessment: Vagus nerve stimulation for epilepsy, A report of the Therapeutics and Technology Assessment Subcommittee of the American Academy of Neurology,” Neurology 1999; 53:666-669. While not highly effective, it has been estimated that VNS reduces seizures by an average of approximately 30-50% in about 30-50% of subjects who are implanted with the device. Unfortunately, a vast majority of the subjects who are outfitted with the VNS device from Cyberonics, Inc., of Houston, Texas, still suffer from un-forewarned seizures and many subjects obtain no benefit whatsoever.

Another recent alternative electrical stimulation therapy for the treatment of epilepsy is deep brain stimulation (DBS). Open-loop deep brain stimulation has been attempted at several anatomical target sites, including the anterior nucleus of the thalamus, the centromedian nucleus of the thalamus, and the hippocampus. The results have shown some potential to reduce seizure frequency, but the efficacy leaves much room for improvement.

Another type of electrical stimulation therapy for the treatment epilepsy has been proposed by NeuroPace, Inc., of Mountain View, Calif., in which an implanted device is designed to detect abnormal electrical activity in the brain and respond by delivering electrical stimulation to the brain. The results of clinical trials of this system have also demonstrated some potential to reduce seizure frequency.

One of the most devastating aspects of epilepsy is the uncertainty of when seizures might occur, an uncertainty that transforms brief episodic events into a debilitating chronic condition. For over 30 years, researchers have tried to reduce this uncertainty by identifying electroencephalogram (EEG) signals that would predict the occurrence of a seizure. There have been a number of proposals described in the patent literature regarding the use of predictive algorithms that purportedly can predict the onset of a seizure. When the predictive algorithm predicts the onset of a seizure, some type of warning is provided to the subject regarding the oncoming seizure or some sort of therapy is initiated. For example, U.S. Pat. Nos. 3,863,625 to Viglione, 5,995,868 to Dorfmeister et al., and 6,658,287 to Litt et al., describe a variety of proposed seizure prediction systems. However, to date, none of the proposed seizure prediction systems have shown statistically significant results.

The temporal progression of a seizure may be described in terms of intervals or states: interictal, pro-ictal (including pre-ictal), ictal, and postictal. The interictal state is comprised of relatively normative EEG that represents the state in between seizures. The ictal state refers to the state during which there is seizure activity. The postictal state is the state immediately following a seizure or ictal state.

The pro-ictal state represents a state of high susceptibility for seizure; in other words, a seizure can happen at any time. Some researchers have proposed that seizures develop minutes to hours before the clinical onset of the seizure. These researchers therefore classify a pre-ictal condition as the beginning of the ictal or seizure event which begins with a cascade of events. Under this definition, a seizure is imminent and will occur if a pre-ictal condition is observed. Others believe that a pre-ictal condition represents a state which only has a high susceptibility for a seizure and does not always lead to a seizure and that seizures occur either due to chance (e.g., noise) or via a triggering event during this high susceptibility time period. For clarity, the term “pro-ictal” is used herein to describe a general state or condition during which the patient has a high susceptibility for seizure. Accordingly, the pre-ictal state as used in either definition above would be considered to be a pro-ictal state. The EEG characteristics indicative of a pro-ictal interval are not fully understood, but many characteristics have been hypothesized. These include increased spatial synchrony or coherence, localized entrainment of dynamic properties, and changes in EEG amplitude distributions or spectral distributions. If a transition from pro-ictal interval to ictal (seizure) interval occurs, it is in turn followed by a postictal interval characterized by suppression and slowing of the EEG.

While being able to determine that the patient is in a pro-ictal condition is highly desirable, identifying when the patient has entered or is likely to enter a pro-ictal condition is only part of the solution for these patients. An equally important aspect of any seizure advisory system is the ability to inform the patient when they are unlikely to have a seizure for a predetermined period of time (e.g., when the patient has a low susceptibility of seizure or is in a “contra-ictal” state). A more detailed discussion of the identification and indication of a contra-ictal condition may be found in commonly-owned U.S. patent application Ser. No. 12/020,450, filed Jan. 25, 2008, published as Publication No. 2008/0183096, the disclosure of which is incorporated by reference herein in its entirety.

The effort to develop seizure advisory technology has been hampered by limitations of data recording equipment, inadequate computing power, small/incomplete datasets, and lack of rigorous statistical analysis. With regards to statistical analysis, a majority of published work has suffered from one or more of the following problems: (1) lack of statistical power, primarily due to inadequate interictal EEG; (2) absence of a statistical control, e.g. chance predictor; (3) use of a posteriori information in the assessment of algorithm performance, including the use of in-sample data for algorithm testing, and retrospective selection of data channels (electrodes) for best performance; (4) lack of complete performance characterization: sensitivity, specificity, negative predictive value, positive predictive value; and (5) inclusion of clustered seizures in sensitivity analysis, despite the lack of statistical independence and intervening interictal condition.

Many of these shortcomings were recently catalogued in a review of more than 40 seizure prediction studies, in which the authors conclude that “the current literature allows no definite conclusion as to whether seizures are predictable by prospective algorithms.” See Mormann et al., “Seizure prediction: the long and winding road,” BRAIN, vol. 130, no. 2, pp. 314-333, 28 Sept 2006.

One approach for an EEG analysis algorithm within a patient seizure advisory system is to train the algorithm using all electrographic seizure data within the dataset, irrespective of the kind of seizure (clinical or subclinical) or the particular seizure onset characteristics (spatial and temporal pattern at seizure onset). See, e.g., commonly-owned U.S. Patent Publication No. 2008/0208074, filed Feb. 21, 2008, the disclosure of which is incorporated by reference herein in its entirety. Devices employing such algorithms would advise of both clinical and subclinical seizures, with the subclinical seizure warnings possibly being perceived as false positives. In addition, the device might be unable to distinguish one seizure onset characteristic from another.

### SUMMARY OF THE INVENTION

Described herein are methods of developing a brain state analysis system using subject EEG data that distinguishes clinical from subclinical electrographic seizures and, optionally, that distinguishes among different seizure onset characteristics. An algorithm trained on only clinical electrographic seizures would predict clinical seizures more accurately with fewer perceived false positives. In addition, algorithms trained on a particular onset condition may distinguish and advise on that onset condition when used by the patient. The invention provides a brain state system and method of treating a subject using algorithms developed in this manner.

A method of developing a brain state advisory system is provided, comprising: deriving a brain state advisory algorithm and placing the advisory algorithm in memory of the brain state advisory system. The deriving step comprises: analyzing patient EEG data, identifying within the EEG data pro-ictal states correlated with clinical electrographic seizures, and generating pro-ictal state alerts corresponding to pro-ictal states preferentially correlated with clinical electrographic seizures over pro-ictal states correlated with subclinical electrographic seizures.

A brain state system is provided, comprising: an advisory system having a controller programmed to generate a pro-ictal state alert preferentially correlated with clinical electrographic seizures over subclinical electrographic seizures; and an alert indicator communicating with the controller to indicate the pro-ictal state alert.

A method of treating a subject is provided, comprising: obtaining an EEG dataset from the subject; identifying a pro-ictal state preferentially correlated with a clinical electrographic seizure over a subclinical electrographic seizure; and generating a pro-ictal state alert corresponding to the pro-ictal state identified in the identifying step.

A method of developing a seizure prediction system is provided, comprising: analyzing a patient EEG data set including clinical electrographic seizures and subclinical electrographic seizures; and developing a seizure prediction algorithm for predicting seizures based on brain states preferentially correlated with clinical electrographic seizures over subclinical electrographic seizures.

### BRIEF DESCRIPTION OF THE DRAWINGS

FIG. 1 is an analysis showing sensitivity over that of a chance predictor when training on correlated clinical seizures (CCS) and clinical equivalent seizures (CES), and scoring on CCS and CES, compared to training on CCS and CES and scoring on non-clinical seizures (NCS).

FIG. 2 is an analysis showing sensitivity over that of a chance predictor when training on NCS and scoring on NCS, compared to training on NCS and scoring on CCS and CES.

FIG. 3 is an analysis showing sensitivity over that of a chance predictor when training on a first onset characteristic (OC1) and scoring on OC1, compared to training on OC1 and scoring on the remainder of onset characteristics.

FIG. 4 is an analysis showing sensitivity over that of a chance predictor when training on a second onset characteristic (OC2) and scoring on OC2, compared to training on OC2 and scoring on the remainder of onset characteristics.

FIG. 5 illustrates an exemplary embodiment of a either a data collection system or monitoring system.

FIG. 6 depicts a block diagram example of the overall structure of a system for performing substantially real-time assessment of the subject's brain activity and for determining the communication output that is provided to the subject or caregiver.

FIG. 7 illustrates a method of using the systems described herein to collect data, tune the algorithms, and use the tuned algorithms to estimate the subject's susceptibility to a seizure.

FIG. 8 illustrates a system including a closed-loop therapy delivery assembly.

FIG. 9 is a histogram showing the percentage of CCS's that make up their dominant onset characteristic type.

### DETAILED DESCRIPTION OF THE INVENTION

Certain specific details are set forth in the following description and figures to provide an understanding of various embodiments of the invention. Certain well-known details, associated electronics and devices are not set forth in the following disclosure to avoid unnecessarily obscuring the various embodiments of the invention. Further, those of ordinary skill in the relevant art will understand that they can practice other embodiments of the invention without one or more of the details described below. Finally, while various processes are described with reference to steps and sequences in the following disclosure, the description is for providing a clear implementation of particular embodiments of the invention, and the steps and sequences of steps should not be taken as required to practice this invention.

The term “condition” is used herein to generally refer to the subject's underlying disease or disorder—such as epilepsy, depression, Parkinson's disease, headache disorder, etc. The term “state” is used herein to generally refer to calculation results or indices that are reflective a categorical approximation of a point (or group of points) along a single or multi-variable state space continuum of the subject's condition. The estimation of the subject's state does not necessarily constitute a complete or comprehensive accounting of the subject's total situation. As used in the context of the present invention, state typically refers to the subject's state within their neurological condition. For example, for a subject suffering from an epilepsy condition, at any point in time the subject may be in a different states along the continuum, such as an ictal state (a state in which a neurological event, such as a seizure, is occurring), a pro-ictal state (a state in which the subject has an increased risk of transitioning to the ictal state), an inter-ictal state (a state in between ictal states), a contra-ictal state (a state in which the subject has a low risk of transitioning to the ictal state within a calculated or predetermined time period), or the like. A pro-ictal state may transition to either an ictal or inter-ictal state.

The estimation and characterization of state may be based on one or more subject dependent parameters from the a portion of the subject's body, such as electrical signals from the brain, including but not limited to electroencephalogram signals and electrocorticogram signals “ECoG” or intracranial EEG (referred to herein collectively as EEG″), brain temperature, blood flow in the brain, concentration of AEDs in the brain or blood, changes thereof, etc.

An “event” is used herein to refer to a specific event in the subject's condition. Examples of such events include transition from one state to another state, e.g., an electrographic onset of seizure, end of seizure, or the like. For conditions other than epilepsy, the event could be an onset of a migraine headache, onset of a depressive episode, a tremor, or the like.

The occurrence of a seizure may be referred to as a number of different things. For example, when a seizure occurs, the subject is considered to have exited a “pro-ictal state” and has transitioned into the “ictal state”. However, the electrographic onset of the seizure (one event) and/or the clinical onset of the seizure (another event) have also occurred during the transition of states.

The devices and systems of the present invention can be used for long-term, ambulatory sampling and analysis of one or more physiological signals, such as a subject's brain activity (e.g., EEG). In many embodiments, the systems and methods of the present invention incorporate brain activity analysis algorithms that extract one or more features from the brain activity signals (and/or other physiological signals) and classifies, or otherwise processes, such features to determining the subject's susceptibility for having a seizure.

Some systems of the present invention may also be used to facilitate delivery of a therapy to the subject to prevent the onset of a seizure and/or abort or mitigate a seizure. Facilitating the delivery of the therapy may be carried out by outputting a warning or instructions to the subject or automatically initiating delivery of the therapy to the subject (e.g., pharmacological, electrical stimulation, focal cooling, etc.). The therapy may be delivered to the subject using an implanted assembly that is used to collect the ambulatory signals, or it may be delivered to the subject through a different implanted or external assembly.

A more detailed description of systems and algorithms that may be used to deliver a therapy to the subject are described in commonly owned U.S. Pat. Nos. 6,366,813, filed Jun. 25, 1999; 6,819,956, filed Nov. 11, 2001; 7,209,787, filed Nov. 20, 2003; 7,242,984, filed Jan. 6, 2004; 7,277,758, filed Apr. 5, 2004; 7,231,254, filed Jul. 12, 2004; 7,403,820, filed May 25, 2005; 7,324,851, filed Jun. 1, 2004; and 7,623,928, filed May 2, 2007; and U.S. patent application Ser. Nos. 11/321,897, filed Dec. 28, 2005; 11/321,898, filed Dec. 28, 2005; 11/322,150, filed Dec. 28, 2005; 11/766,742, filed Jun. 21, 2007; 11/766,751, filed Jun. 21, 2007; 11/766,756, filed Jun. 21, 2007; 11/766,760, filed Jun. 21, 2007; 12/020,507, filed Jan. 25, 2008; 11/599,179, filed Nov. 14, 2006; 12/053,312, filed Mar. 21, 2008; 12/020,450, filed Jan. 25, 2008; 12/035,335, filed Feb. 21, 2008; and 12/180,996, filed Jul. 28, 2008; the complete disclosures of which are incorporated herein by reference in their entireties.

For subjects suspected or known to have epilepsy, the systems described herein may be used to collect data and quantify metrics for the subjects who heretofore have not been accurately measurable. For example, the data may be analyzed to (1) determine whether or not the subject has epilepsy, (2) determine the type of epilepsy, (3) determine the types of seizures, (4) localize or lateralize one or more seizure foci or seizure networks, (5) assess baseline seizure statistics and/or change from the baseline seizure statistics (e.g., seizure count, frequency, duration, seizure pattern, etc.), (6) monitor for sub-clinical seizures, assess a baseline frequency of occurrence, and/or change from the baseline occurrence, (7) measure the efficacy of AED treatments, deep brain or cortical stimulation, peripheral nerve stimulation, and/or cranial nerve stimulation, (8) assess the effect of adjustments of the parameters of the AED treatment, (9) determine the effects of adjustments of the type of AED, (10) determine the effect of, and the adjustment to parameters of, electrical stimulation (e.g., peripheral nerve stimulation, cranial nerve stimulation, deep brain stimulation (DBS), cortical stimulation, etc.), (11) determine the effect of, and the adjustment of parameters of focal cooling (e.g., use of cooling fluids, peltier devices, etc., to diminish or reduce seizures (see, for example, “Rothman et al., “Local Cooling: A Therapy for Intractable Neocortical Epilepsy,” Epilepsy Currents, Vol. 3, No. 5, September/October 2003; pp. 153-156), (12) determine “triggers” for the subject's seizures, (13) assess outcomes from surgical procedures, (14) provide immediate biofeedback to the subject, (15) screen subjects for determining if they are an appropriate candidate for a seizure advisory system or other neurological monitoring or therapy system, or the like.

In a first aspect of the invention, the system encompasses a data collection system that is adapted to collect long term ambulatory brain activity data from the subject. In preferred embodiments, the data collection system is able to sample one or more channels of brain activity from the subject with one or more implanted electrodes. The electrodes are in wired or wireless communication with one or more implantable assemblies that are, in turn, in wired or wireless communication with an external assembly. The sampled brain activity data may be stored in a memory of the implanted assembly, external assembly and/or a remote location such as a physician's computer system. In alternative embodiments, it may be desirable to integrate the electrodes with the implanted assembly, and such an integrated implanted assembly may be in communication with the external assembly.

Unlike other conventional systems which have an implanted memory that is able to only store small epochs of brain activity before and after a seizure, the implantable assemblies of the present invention are configured to substantially continuously sample the physiological signals over a much longer time period (e.g., anywhere between one day to one week, one week to two weeks, two weeks to a month, or more) so as to be able to monitor fluctuations of the brain activity (or other physiological signal) over the entire time period. In alternative embodiments, however, the implantable assembly may only periodically sample the subject's physiological signals or selectively/aperiodically monitor the subject's physiological signals. Some examples of such alternative embodiments are described in commonly owned U.S. patent application Ser. Nos. 11/616,788, filed Dec. 27, 2006, and 11/616,793, filed Dec. 27, 2006, the complete disclosures of which are incorporated herein by reference in their entireties.

When the memory is almost full, the system may provide the subject a warning so that the subject may manually initiate uploading of the collected brain activity data or the system may automatically initiate a periodic download of the collected brain activity data from a memory of the external assembly to a hard drive, flash-drive, local computer workstation, remote server or computer workstation, or other larger capacity memory system. In alternative embodiments, the external assembly may be configured to automatically stream the stored EEG data over a wireless link to a remote server or database. Such a wireless link may use existing WiFi networks, cellular networks, pager networks or other wireless network communication protocols. Advantageously, such embodiments would not require the subject to manually upload the data and could reduce the down time of the system and better ensure permanent capture of substantially all of the sampled data.

The system includes an electrode and an implanted communication assembly in communication with the electrode. The implanted communication assembly samples a neural signal with the electrode and substantially continuously transmits a data signal from the subject's body. The system also comprises an external assembly positioned outside the subject's body that is configured to receive and process the data signal to measure the subject's susceptibility to having a seizure. In alternative embodiments the implanted assembly processes the data and measures the subject's susceptibility of having a seizure, in which case only data indicative of the measured susceptibility is transmitted to the external assembly.

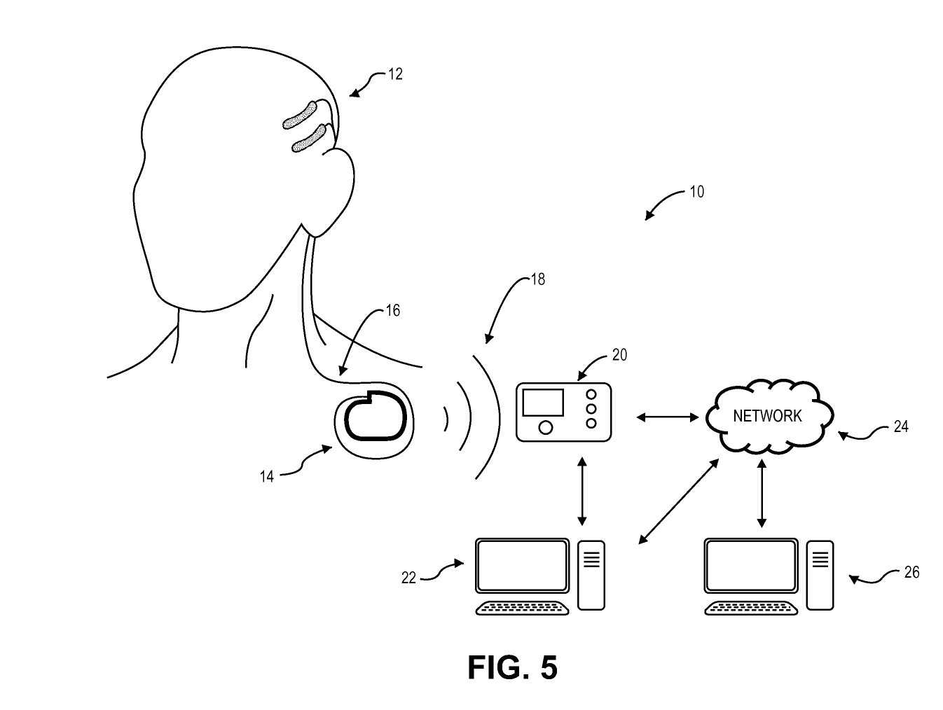

FIG. 5 illustrates an exemplary embodiment of a either a data collection system or monitoring system as described herein. System 10 includes one or more electrode arrays 12 that are configured to be implanted in the subject and configured to sample electrical activity from the subject's brain. The electrode array 12 may be positioned anywhere in, on, and/or around the subject's brain, but typically one or more of the electrodes are implanted within the subject's dura. For example, one of more of the electrodes may be implanted adjacent or above a previously identified epileptic network, epileptic focus or a portion of the brain where the focus is believed to be located. While not shown, it may be desirable to position one or more electrodes in a contralateral position relative to the focus or in other portions of the subject's body to monitor other physiological signals. Other methods for positioning the electrodes are described in commonly-owned co-pending U.S. patent application Ser. No. 12/630,300, filed Dec. 3, 2009, incorporated by reference herein in its entirety.

The electrode arrays 12 of the present invention may be, for example, intracranial electrodes (e.g., epidural, subdural, and/or depth electrodes), extracranial electrodes (e.g., spike or bone screw electrodes, subcutaneous electrodes, scalp electrodes, dense array electrodes), or a combination thereof. While it is preferred to monitor signals directly from the brain, it may also be desirable to monitor brain activity using sphenoidal electrodes, foramen ovale electrodes, intravascular electrodes, peripheral nerve electrodes, cranial nerve electrodes, or the like.

In the configuration illustrated in FIG. 5, two electrode arrays 12 are positioned in an epidural or subdural space, but as noted above, any type of electrode placement may be used to monitor brain activity of the subject. For example, in a minimally invasive embodiment, the electrode array 12 may be implanted between the skull and any of the layers of the scalp. Specifically, the electrodes 12 may be positioned between the skin and the connective tissue, between the connective tissue and the epicranial aponeurosis/galea aponeurotica, between the epicranial aponeurosis/galea aponeurotica and the loose aerolar tissue, between the loose aerolar tissue and the pericranium, and/or between the pericranium and the calvarium. To improve signal-to-noise ratio, such subcutaneous electrodes may be rounded to conform to the curvature of the outer surface of the cranium, and may further include a protuberance that is directed inwardly toward the cranium to improve sampling of the brain activity signals. Furthermore, if desired, the electrode may be partially or fully positioned in openings disposed in the skull. Additional details of exemplary wireless minimally invasive implantable devices and their methods of implantation can be found in U.S. patent application Ser. No. 11/766,742, filed Jun. 21, 2007, published as Publ. No. 2008/0027515, the disclosure of which is incorporated by reference herein in its entirety.

As shown in FIG. 5, the electrode arrays 12 are in wired communication with an implanted assembly 14 via the wire leads 16. The individual leads from the contacts (not shown) are placed in lead 16 and the lead 16 is tunneled between the cranium and the scalp and subcutaneously through the neck to the implanted assembly 14. Typically, implanted assembly 14 is implanted in a sub-clavicular pocket in the subject, but the implanted assembly 14 may be disposed somewhere else in the subject's body. For example, the implanted assembly 14 may be implanted in the abdomen or underneath, above, or within an opening in the subject's cranium (not shown). Further details of exemplary systems may be found in U.S. patent application Ser. No. 12/020,507, filed Jan. 25, 2008, published as Publ. No. 2008/0183097, the disclosure of which is incorporated by reference herein in its entirety.

Implanted assembly 14 can be used to pre-process EEG signals sampled by the electrode array 12 and transmit a data signal that is encoded with the sampled EEG data over a wireless link 18 to an external assembly 20, where the EEG data is permanently or temporarily stored. The data signals that are wirelessly transmitted from implanted assembly 14 may be encrypted so as to help ensure the privacy of the subject's data prior to transmission to the external assembly 20. Alternatively, the data signals may be transmitted to the external assembly 20 with unencrypted EEG data, and the EEG data may be encrypted prior to the storage of the EEG data in the memory of external assembly 20 or prior to transfer of the stored EEG data to the local computer workstation 22 or remote server 26. Alerts generated by the system may communicated to the subject or to a caregiver via lights or other indicators on the external assembly 20 or via text or graphic communication through workstation 22 or server 26.

If the system includes the capability of providing stimulation of the peripheral nerve, such as the vagus nerve, the system may include a vagus nerve cuff, which includes a connector similar to the IS1 connector that is used for Cyberonics vagus nerve lead. The systems of the present invention may also be configured to provide electrical stimulation to other portions of the nervous system (e.g., cortex, deep brain structures, cranial nerves, etc.). Stimulation parameters are typically about several volts in amplitude, 50 microsec to 1 millisec in pulse duration, and at a frequency between about 2 Hz and about 1000 Hz.

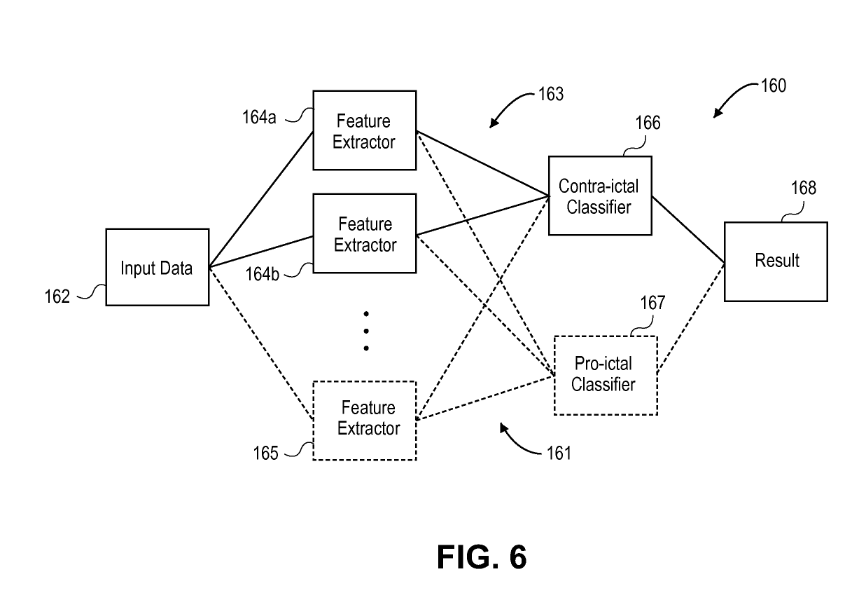

FIG. 6 depicts a block diagram example of the overall structure of a system for performing substantially real-time assessment of the subject's brain activity and for determining the communication output that is provided to the subject or caregiver. A more detailed discussion of such a system may be found in commonly-owned U.S. patent application Ser. No. 12/020,450, filed Jan. 25, 2008, published as Publication No. 2008/0183096, the disclosure of which is incorporated by reference herein in its entirety. The system may comprise one or more algorithms or modules that process input data 162. The algorithms may take a variety of different forms, but typically comprises one or more feature extractors 164a, 164b, 165 and at least one classifier 166 and 167. The embodiment illustrated in FIG. 6 shows a contra-ictal algorithm 163 and a pro-ictal algorithm 161 which share at least some of the same feature extractors 164a and 164b. In alternative embodiments, however, the algorithms used in the system may use exactly the same feature extractors or completely different feature extractors.

The input data 162 is typically EEG, but may comprise representations of physiological signals obtained from monitoring a subject and may comprise any one or combination of the aforementioned physiological signals from the subject. The input data may be in the form of analog signal data or digital signal data that has been converted by way of an analog to digital converter (not shown). The signals may also be amplified, preprocessed, and/or conditioned to filter out spurious signals or noise. For purposes of simplicity the input data of all of the preceding forms is referred to herein as input data 162. In one preferred embodiment, the input data comprises between about 1 channel and about 64 channels of EEG from the subject.

The input data 162 from the selected physiological signals is supplied to the one or more feature extractors 164a, 164b, 165. Feature extractor 164a, 164b, 165 may be, for example, a set of computer executable instructions stored on a computer readable medium, or a corresponding instantiated object or process that executes on a computing device. Certain feature extractors may also be implemented as programmable logic or as circuitry. In general, feature extractors 164a, 164b, 165 can process data 162 and identify some characteristic of interest in the data 162. Such a characteristic of the data is referred to herein as an extracted feature.

Each feature extractor 164a, 164b, 165 may be univariate (operating on a single input data channel), bivariate (operating on two data channels), or multivariate (operating on multiple data channels). Some examples of potentially useful characteristics to extract from signals for use in determining the subject's propensity for a neurological event, include but are not limited to, bandwidth limited power (alpha band [8-13 Hz], beta band [13-18 Hz], delta band [0.1-4 Hz], theta band [4-8 Hz], low beta band [12-15 Hz], mid-beta band [15-18 Hz], high beta band [18-30 Hz], gamma band [30-48 Hz], high frequency power [>48 Hz], bands with octave or half-octave spacings, wavelets, etc.), second, third and fourth (and higher) statistical moments of the EEG amplitudes or other features, spectral edge frequency, decorrelation time, Hjorth mobility (HM), Hjorth complexity (HC), the largest Lyapunov exponent L(max), effective correlation dimension, local flow, entropy, loss of recurrence LR as a measure of non-stationarity, mean phase coherence, conditional probability, brain dynamics (synchronization or desynchronization of neural activity, STLmax, T-index, angular frequency, and entropy), line length calculations, first, second and higher derivatives of amplitude or other features, integrals, and mathematical linear and non-linear operations including but not limited to addition, subtraction, division, multiplication and logarithmic operations. Of course, for other neurological conditions, additional or alternative characteristic extractors may be used with the systems described herein.

The extracted characteristics can be supplied to the one or more classifiers 166, 167. Like the feature extractors 164a, 164b, 165, each classifier 166, 167 may be, for example, a set of computer executable instructions stored on a computer readable medium or a corresponding instantiated object or process that executes on a computing device. Certain classifiers may also be implemented as programmable logic or as circuitry.

The classifiers 166, 167 analyze one or more of the extracted characteristics, and either alone or in combination with each other (and possibly other subject dependent parameters), provide a result 168 that may characterize, for example, a subject's condition. The output from the classifiers may then be used to determine the subject's susceptibility for having a seizure, which can determine the output communication that is provided to the subject regarding their condition. As described above, the classifiers 166, 167 are trained by exposing them to training measurement vectors, typically using supervised methods for known classes, e.g. ictal, and unsupervised methods as described above for classes that can't be identified a priori, e.g. contra-ictal. Some examples of classifiers include k-nearest neighbor (“KNN”), linear or non-linear regression, Bayesian, mixture models based on Gaussians or other basis functions, neural networks, and support vector machines (“SVM”). Each classifier 166, 167 may provide a variety of output results, such as a logical result or a weighted result. The classifiers 166, 167 may be customized for the individual subject and may be adapted to use only a subset of the characteristics that are most useful for the specific subject. Additionally, over time, the classifiers 166, 167 may be further adapted to the subject, based, for example, in part on the result of previous analyses and may reselect extracted characteristics that are used for the specific subject.

For the embodiment of FIG. 6, the pro-ictal classifier 167 may classify the outputs from feature extractors 164a, 164b to detect characteristics that indicate that the subject is at an elevated susceptibility for a neurological event, while the contra-ictal classifier 166 may classify the outputs from feature extractors 164a, 164b, 165 to detect characteristics that occur when the subject is unlikely to transition into an ictal condition for a specified period of time. The combined output of the classifiers 166, 167 may be used to determine the output communication provided to the subject. In embodiments which comprise only the contra-ictal algorithm, the output from the contra-ictal classifier 166 alone may be used to determine the output communication to the subject.

Depending on the specific feature extractors and classifiers used, the computational demands of the analysis provided by feature extractors 164a, 164b, 165 and classification provided by classifiers 166, 167 can be extensive. In the case of ambulatory systems supplied by portable power sources, such as batteries, supplying the power required to meet the computational demands can severely limit power source life. In preferred embodiments, both the seizure advisory algorithm are embodied in the external assembly 20. Processing the EEG data with the algorithms in the external assembly 20 provides a number of advantages over having the algorithms in the implanted assembly. First, keeping the processing in the external assembly 20 will reduce the overall power consumption in the implanted assembly 14 and will prolong the battery life of the implanted assembly 14. Second, charging of battery or replacing the battery of the external assembly 20 is much easier to accomplish. The battery of the external assembly may be charged by placing the external assembly 20 in a recharging cradle (e.g., inductive recharging) or simply by attaching the external assembly to an AC power source. Third, customizing, tuning and/or upgrading the algorithms will be easier to achieve in the external assembly 20. Such changes may be carried out by simply connecting the external assembly to the physician's computer workstation 20 and downloading the changes. Alternatively, upgrading may be performed automatically over a wireless connection with the communication sub-assembly 64.

While it is preferred to have the observer algorithms 160 in the external assembly 20, in alternate embodiments of the present invention, the observer algorithms 160 may be wholly embodied in the implanted assembly 14 or a portion of one or more of the observer algorithms 160 may be embodied in the implanted assembly 14 and another portion of the one or more algorithms may be embodied in the external assembly 20. In such embodiments, the processing sub-assembly 44 (or equivalent component) of the implanted assembly 14 may execute the analysis software, such as a seizure advisory algorithm(s) or portions of such algorithms. For example, in some configurations, one or more cores of the processing sub-assembly 44 may run one or more feature extractors that extract features from the EEG signal that are indicative of the subject's susceptibility to a seizure, while the classifier could run on a separate core of the processing sub-assembly 44. Once the feature(s) are extracted, the extracted feature(s) may be sent to the communication sub-assembly 46 for the wireless transmission to the external assembly 20 and/or store the extracted feature(s) in memory sub-system 52 of the implanted assembly 14. Because the transmission of the extracted features is likely to include less data than the EEG signal itself, such a configuration will likely reduce the bandwidth requirements for the wireless communication link 18 between the implantable assembly 14 and the external assembly 20.

In other embodiments, the seizure advisory algorithms may be wholly embodied within the implanted assembly 14 and the data transmission to the external assembly 29 may include the data output from the classifier, a warning signal, recommendation, or the like. A detailed discussion of various embodiments of the internal/external placement of such algorithms are described in commonly owned U.S. Patent Application Publ. No. 2007/0149952 and 2008/0027348, the complete disclosures of which are incorporated herein by reference in its entirety.

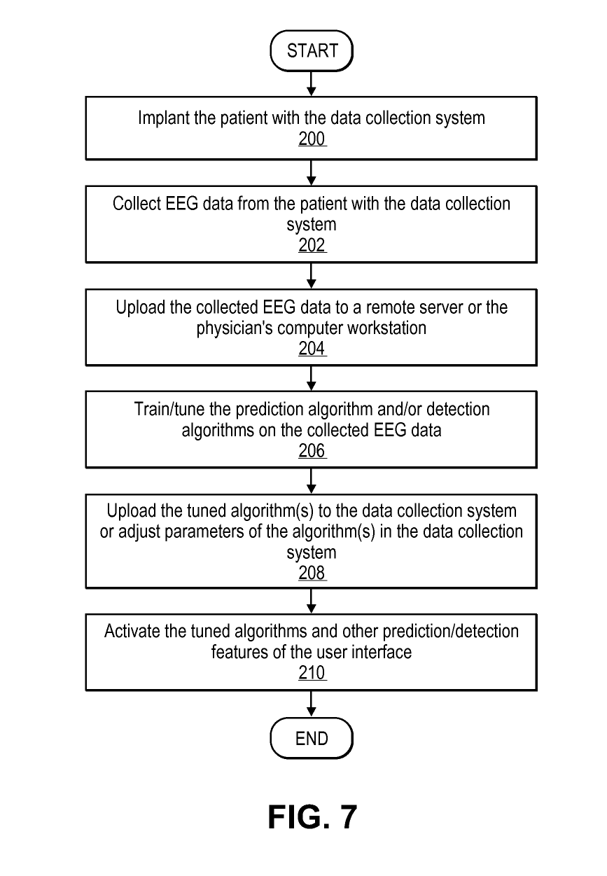

FIG. 7 illustrates a method of using the systems described herein to collect data, tune the algorithms, and use the tuned algorithms to estimate the subject's susceptibility to a seizure. At step 200, the subject is implanted with the system 10 in which the seizure advisory algorithms are disabled or not yet present in the system. The user interface aspects that are related to the seizure advising may also be disabled.

At step 202, the system is used to collect EEG data for a desired time period, as described in detail above. Generally, the desired time period will be a specified time period such as at least one week, between one week and two weeks, between two weeks and one month, between one month and two months, or two months or more. But the desired time period may simply be a minimum time period that provides a desired number of seizure events. At step 204, the collected EEG data may be periodically downloaded to the physician's computer workstation or the entire EEG data may be brought into the physician's office in a single visit.

At step 206, the physician may analyze the EEG data using the computer workstation that is running EEG analysis software, the EEG data may be transferred to a remote analyzing facility that comprises a multiplicity of computing nodes where the EEG data may be analyzed on an expedited basis, or it may even be possible to analyze the EEG analysis software in the external assembly 20. Analysis of the EEG data may be performed in a piecewise fashion after the shorter epochs of EEG data are uploaded to the database, or the analysis of the EEG data may be started after the EEG data for the entire desired time period has been collected. Typically, “analysis of the EEG data” will include identifying and annotating at least some of spike, bursts, the earliest electrographic change (EEC), unequivocal electrical onset (UEO), unequivocal clinical onset (UCO), electrographic end of seizure (EES). Identification of such events may be performed automatically with a seizure detection algorithm, manually based on visual inspection by a human (e.g., by board certified epileptologists), or a combination thereof. After the EEG data is annotated, the seizure advisory algorithm(s) may be trained on the annotated EEG data in order to tune the parameters of the algorithm(s) to the subject specific EEG data.

Once the algorithm(s) are tuned to meet minimum performance criteria, at step 208 the tuned algorithm(s) or the parameter changes to the base algorithm may be uploaded to the external assembly 20. At step 210, the tuned algorithm and the other user interface aspects of the present invention may be activated, and the observer algorithm may be used by the subject to monitor the subject's susceptibility to a seizure and/or detect seizures.

When the seizure advisory system 10 determines that the subject is at an increased susceptibility to a seizure (or otherwise detects a seizure), the external assembly may be configured to generate a seizure warning to the subject, as described above. For example, the external assembly may activate a red or yellow LED light, generate a visual warning on the LCD, provide an audio warning, deliver a tactile warning, or any combination thereof. If desired, the warning may be “graded” so as to indicate the confidence level of the seizure advisory, indicate the estimated time horizon until the seizure, or the like. “Grading” of the warning may be through generation of different lights, audio, or tactile warning or a different pattern of lights, audio or tactile warnings.

Additionally or alternatively, the external assembly may include an instruction to the subject regarding an appropriate therapy for preventing or reducing the susceptibility for the seizure. The instruction may instruct the subject to take a dosage of their prescribed AED, perform biofeedback to prevent/abort the seizure, manually activate an electrical stimulator (e.g., use a wand to activate an implanted VNS device) or merely to instruct the subject to make themselves safe. A more complete description of various instructions that may be output to the subject are described in commonly owned, copending U.S. patent application Ser. Nos. 11/321,897, filed Dec. 28, 2005, and 11/321,898, filed Dec. 28, 2005, both of which are incorporated by reference herein in their entireties.

The outputs provided to the subject via the external assembly may be a standardized warning or instruction, or it may be programmed by the physician to be customized specifically to the subject and their condition. For example, different subjects will be taking different AEDs, different dosages of the AEDs, and some may be implanted with manually actuatable stimulators (e.g., NeuroPace RNS, Cyberonics VNS, etc.), and the physician will likely be desirous to customize the therapy to the subject. Thus, the physician will be able to program the warning and/or instruction to correspond to the level of susceptibility, estimated time horizon to seizure, or the like.

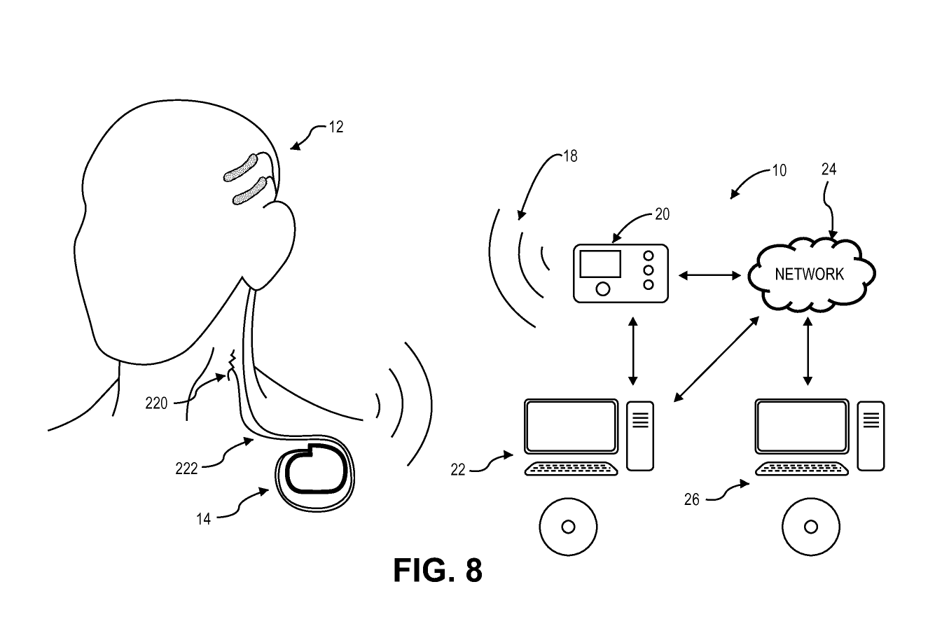

The systems of the present invention may also be adapted to provide therapy to the subject. FIG. 8 illustrates one embodiment of the system 10 that includes closed-loop therapy delivery assembly in the implanted assembly 14. The system 10 illustrated in FIG. 8 will generally have the same components as shown in FIG. 5, but will also include an implanted pulse generator (not shown) that is in communication with a vagus nerve cuff electrode 220 via a lead 222. When the seizure advisory system determines that the subject is at an elevated susceptibility to a seizure, the system may automatically initiate delivery of electrical stimulation to the vagus nerve cuff electrode. The parameters (e.g., burst/no burst mode, amplitude, pulse width, pulse frequency, etc.) of the electrical stimulation may be varied based on the subject's susceptibility, or the parameter may be constant.

While not shown in FIG. 8, the present invention further embodies other therapy outputs—such as electrical stimulation of the brain tissue (e.g., deep brain structures, cortical stimulation) using electrode array 12 or other electrode arrays (not shown), stimulation of cranial nerves (e.g., trigeminal stimulation), delivery of one or more drugs via implanted drug dispensers, cryogenic therapy to the brain tissue, cranial nerves, and/or peripheral nerves), or the like. Similar to vagus nerve stimulation, parameters of the therapy may be constant or the parameters of the therapy may be modified based on the subject's estimated susceptibility.

According to one aspect of the invention, the system's seizure advisory algorithm may be trained to distinguish clinical from subclinical electrographic seizures and, optionally, that distinguishes among different seizure onset characteristics. The following discussion describes a method of developing such a seizure advisory system and examples using actual subject EEG data.

The inventors added the following annotations to subject EEG records:

(a) Correlated clinical seizure (CCS)—An electrographic seizure with physiological accompaniment, as confirmed by site annotation, chart notes, or video.

(b) Clinical equivalent seizure (CES)—An electrographic seizure, in the absence of site annotation, chart notes, or video, that has a high likelihood of manifesting clinically, based upon known CCS characteristics and/or magnitude, propagation, and/or spread.

(c) Non-clinical seizure (NCS)—An electrographic seizure, in the absence of site annotation, chart notes, or video, that has a low likelihood of manifesting clinically, based upon known CCS characteristics and/or magnitude, propagation, and/or spread.

(d) Onset characteristic (OC)—An electrographic seizure labeled with a distinct designator X, that assigns it a unique seizure onset characteristic, indicated by, e.g., waveform, location of focus, unique magnitude, propagation, and/or spread.

Each unequivocal electrographic seizure onset (UEO) was annotated as being a CCS, CES, or NCS and assigned an OC. In alternative embodiments, some or all of the EEG data may be automatically annotated using, e.g., the methods and devices described in commonly owned U.S. patent application Ser. No. 12/343,376, Dec. 23, 2008, the disclosure of which is incorporated herein by reference in its entirety.

Calculations were performed on a specially built multi-node computer network, although any computer or network with sufficient capacity could be used. Classifiers were induced and performance estimated using an epoch-based k-fold cross-validation. All experiments required a minimum of one qualified seizure segment for both training and scoring. The sensitivity over that of a chance predictor, scoring against primary seizures (SnDifference_prim), was the test statistic used in the following analyses outlined in Table 1.

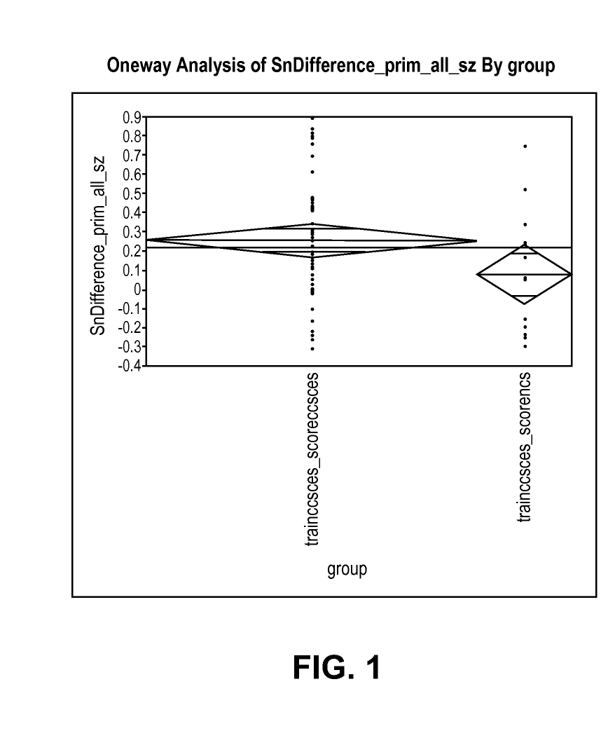

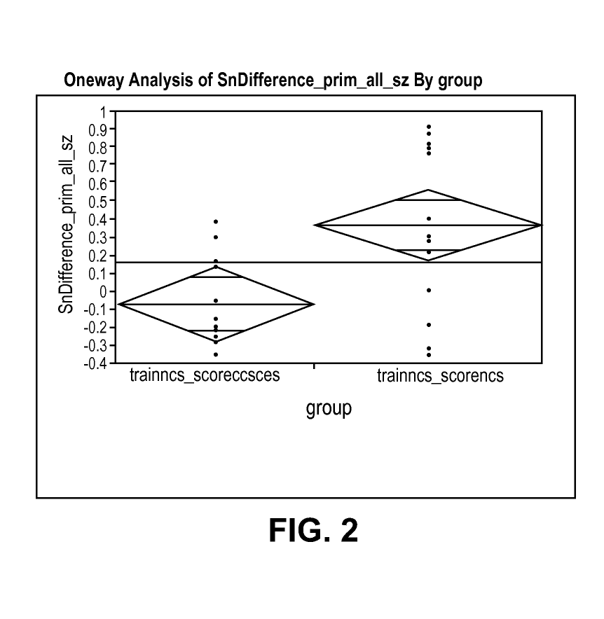

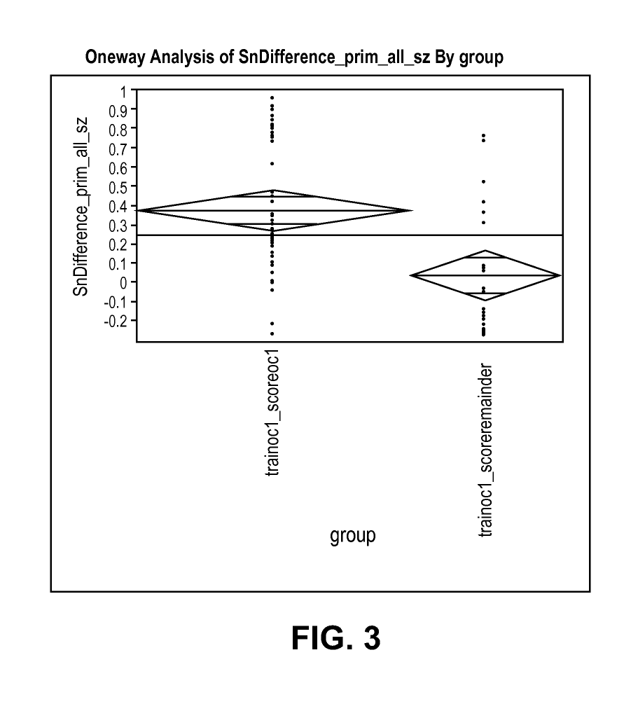

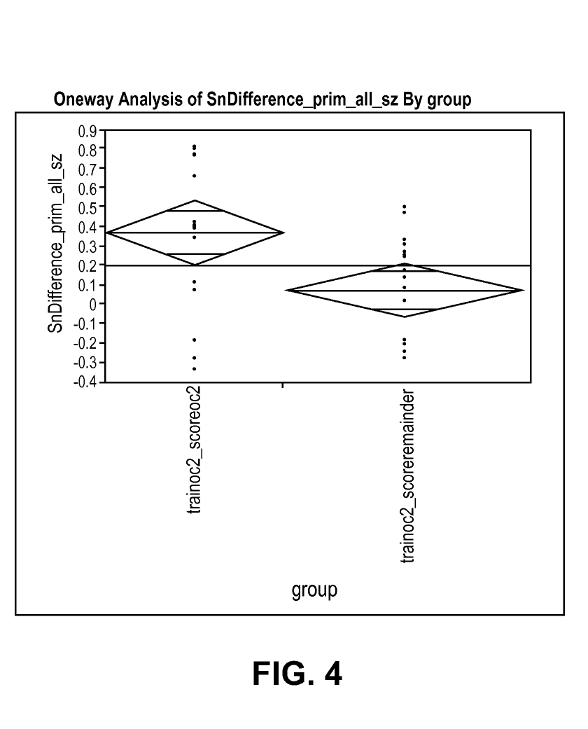

TABLE 1
Results summary

| | Mean | | Analysis |
|---|---|---|---|
| Experiment | SnDifference_prim | Significance | Depicted In: |
| Train CCSCES, Score | 26% vs. 9% | P = 0.03 | FIG. 1 |
| CCSCES better than | | | |
| Train CCSCES, Score | | | |
| NCS | | | |
| Train NCS, Score | 37% vs. −7% | P < 0.01 | FIG. 2 |
| NCS better than | | | |
| Train NCS, Score | | | |
| CCSCES | | | |
| Train OC1, Score | 37% vs. 3% | P < 0.001 | FIG. 3 |
| OC1 better than | | | |
| Train OC1, Score | | | |
| Remainder | | | |
| Train OC2, Score | 37% vs. 7% | P < 0.01 | FIG. 4 |
| OC2 better than | | | |
| Train OC2, Score | | | |
| Remainder | | | |

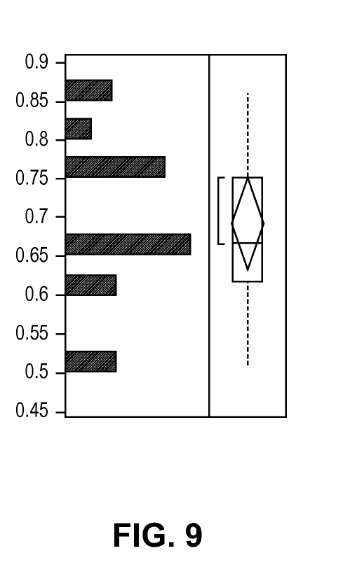

Data used to run all experiments was tabulated as follows:
85.6% of all subjects (N=76) had all of their OC's correlate uniquely with clinical (CCS & CES) or non-clinical (NCS) manifestation.
63.3% of subjects with a CCS (N=44) had a single OC associated with their CCS's. Of the remaining 36.7%, a histogram found in FIG. 9 shows the percentage of CCS's that make up their dominant OC type. Additionally, 65%, 24%, and 11% of those subjects had 2, 3, and 4 OC's, respectively, associated with CCS's.

The following discussion refers to the summary in Table 1. All discussion p values are from the Wilcoxon Rank Sum Test.

Training on CCS's and CES's (FIG. 1) alone provided significantly better (P=0.03) anticipation of CCS's and CES's than NCS's. Conversely, training on NCS's (FIG. 2) alone provided significantly better (P<0.01) better anticipation of NCS's than CCS's and CES's. Likewise, training on OC1 (FIG. 3) alone provided significantly better (P<0.001) anticipation of OC1 than the complement of seizures to OC1. Similarly, training on OC2 (FIG. 4) alone provided significantly better (P<0.01) anticipation of OC2 than the complement of seizures to OC2.

Thus, seizure advisory algorithms may be trained to preferentially anticipate clinical events alone. Separate classifiers for each onset characteristic associated with correlated clinical seizures may also be used. One aspect of our invention therefore provides a method of developing a brain state advisory system by deriving a brain state advisory algorithm to identify within patient EEG data pro-ictal states preferentially correlated with clinical electrographic seizures over subclinical electrographic seizures. Pro-ictal state alerts corresponding to pro-ictal states correlated with clinical electrographic seizures can then be preferentially generated over pro-ictal state alerts corresponding to pro-ictal states correlated with subclinical electrographic seizures. Such an algorithm can then be placed in memory of the brain state advisory system.

Clinical electrographic seizures can be identified either by using primary confirmation of clinical seizure (e.g., annotations by the subject or an observer, chart notes or video) or by an assessment of the EEG data based on known correlated clinical seizure characteristics to identify an EEG waveform that is highly likely to correlate with a clinical manifestation, even in the absence of a chart annotation. Subclinical seizures are seizures corresponding to abnormal brain activity but which do not present any observable clinical signs or symptoms, and may be identified based on analysis of the EEG data.

Seizure advisory algorithms can also be developed by identifying one or more seizure onset characteristics within the EEG data and generating a pro-ictal state alert corresponding to the seizure onset characteristics. The alerts for each seizure onset characteristic may be distinct and unique. The algorithm may also be trained to generate a distinct alert corresponding to a subclinical pro-ictal state, i.e., a pro-ictal state unlikely to manifest clinically. The method may also generate no alerts correlated with subclinical electrographic seizures. In other embodiments, if a pro-ictal state is correlated with both a clinical electrographic seizure and a subclinical electrographic seizure, the method may (1) suppress a pro-ictal state alert or (2) generate only a subclinical pro-ictal state alert.

In accordance with the present invention, a brain state system is provided comprising an advisory system having a controller programmed to generate a pro-ictal state alert preferentially correlated with clinical electrographic seizures over subclinical electrographic seizures; and an alert indicator communicating with the controller to indicate the pro-ictal state alert. The controller may be incorporated into, for example, the implanted assembly 14 or the external assembly 20 shown in FIG. 5, or may be provided as part of a separate component of the system. The system's algorithm may be configured to provide a first pro-ictal state alert corresponding to a first seizure onset characteristic and a second pro-ictal state alert corresponding to a second seizure onset characteristic, with the second pro-ictal state alert being distinct from the first pro-ictal state alert. The system may also be programmed to generate a subclinical pro-ictal state alert correlated with subclinical electrographic seizures and distinct from the pro-ictal state alert corresponding to pro-ictal states correlated with clinical electrographic seizures. The system may also be programmed to generate no pro-ictal state alerts correlated with subclinical electrographic seizures.

The brain state system may also have a therapy system communicating with the controller to provide therapy in response to an alert generated by the advisory system. The therapy may be adapted to provide distinct therapies in response to alerts corresponding to distinct seizure onset characteristics and/or to provide distinct therapies in response to alerts correlated with clinical and subclinical electrographic seizures. In other embodiments, the brain state system may not provide any type of patient advisory or warning. Instead, the brain state system may trigger a therapy in response to a brain state likely to result in a seizure.

In other embodiments, a method of treating a subject is provided. The method includes obtaining an EEG dataset from the subject; identifying a pro-ictal state preferentially correlated with a clinical electrographic seizure over a subclinical electrographic seizure; and generating a pro-ictal state alert corresponding to the identified pro-ictal state. This method may identify a pro-ictal state corresponding with one or more seizure onset characteristics and generate a pro-ictal state alert corresponding to the seizure onset characteristics. The alerts may be distinct and unique.

In addition, the method may also identify a pro-ictal state correlated with a subclinical electrographic seizure and generate a subclinical pro-ictal state alert corresponding to the pro-ictal state correlated with the subclinical electrographic seizure. The subclinical alert may be different and distinct from the clinical seizure alert. Alternatively, the method may generate no pro-ictal alerts correlated with subclinical electrographic seizures.

In some embodiments, therapy may be provided to the subject automatically in response to the alert, and in the case of distinct alerts for different onset conditions or for clinical and subclinical seizures, different therapies may be provided corresponding with the different alerts.

What is claimed is:

1. A method of developing a brain state advisory system, comprising:
deriving a brain state advisory algorithm, the deriving step comprising:
analyzing patient EEG data,
identifying within the EEG data pro-ictal states correlated with clinical electrographic seizures, and
generating pro-ictal state alerts corresponding to pro-ictal states preferentially correlated with clinical electrographic seizures over pro-ictal states correlated with subclinical electrographic seizures;
and
placing the advisory algorithm in memory of the brain state advisory system.

2. The method of claim 1 wherein the identifying step comprises comparing EEG data with primary confirmation of clinical seizure.

3. The method of claim 1 wherein the identifying step comprises comparing EEG data with known EEG data seizure characteristics.

4. The method of claim 1 wherein the identifying step comprises identifying a seizure onset characteristic and the generating step comprises generating a pro-ictal state alert corresponding to the seizure onset characteristic.

5. The method of claim 4 wherein the seizure onset characteristic comprises a first seizure onset characteristic and the identifying step comprises identifying a second seizure onset characteristic and the generating step further comprises generating a pro-ictal state alert corresponding to the second seizure onset characteristic.

6. The method of claim 5 wherein the pro-ictal state alert corresponding to the second seizure onset characteristic is distinct from the pro-ictal state alert corresponding to the first seizure onset characteristic.

7. The method of claim 1 further comprising identifying within the EEG data pro-ictal states correlated with subclinical electrographic seizures and generating subclinical pro-ictal state alerts distinct from the pro-ictal state alerts corresponding to pro-ictal states correlated with clinical electrographic seizures.

8. The method of claim 1 further comprising identifying within the EEG data a pro-ictal state correlated with a subclinical electrographic seizure and suppressing a pro-ictal state alert if a pro-ictal state is also correlated with a clinical electrographic seizure within the same EEG data.

9. The method of claim 1 further comprising identifying within the EEG data a pro-ictal state correlated with a subclinical electrographic seizure and generating a subclinical pro-ictal state alert distinct from the pro-ictal state alerts if a pro-ictal state is also correlated with a clinical electrographic seizure within the same EEG data.

10. The method of claim 1 further comprising generating no pro-ictal alerts correlated with subclinical electrographic seizures.

11. A brain state system comprising:
an advisory system having a controller programmed to generate a pro-ictal state alert preferentially correlated with clinical electrographic seizures over subclinical electrographic seizures; and
an alert indicator communicating with the controller to indicate the pro-ictal state alert.

12. The brain state system of claim 11 wherein the pro-ictal state alert is a first pro-ictal state alert corresponding to a first seizure onset characteristic, wherein the controller is programmed to generate a second pro-ictal state alert corresponding to a second seizure onset characteristic, the second pro-ictal state alert being distinct from the first pro-ictal state alert.

13. The brain state system of claim 11 wherein the controller is programmed to generate a subclinical pro-ictal state alert correlated with subclinical electrographic seizures and distinct from the pro-ictal state alert corresponding to pro-ictal states correlated with clinical electrographic seizures.

14. The brain state system of claim 11 wherein the controller is programmed to generate no pro-ictal state alerts correlated with subclinical electrographic seizures.

15. The brain state system of claim 11 further comprising a patient therapy system communicating with the controller to provide therapy in response to an alert generated by the advisory system.

16. The brain state system of claim 15 wherein the patient therapy system is adapted to provide distinct therapies in response to alerts corresponding to distinct seizure onset characteristics.

17. The brain state system of claim 15 wherein the patient therapy system is adapted to provide distinct therapies in response to alerts correlated with clinical and subclinical electrographic seizures.

18. A method of treating a subject comprising:
obtaining an EEG dataset from the subject;
identifying a pro-ictal state preferentially correlated with a clinical electrographic seizure over a subclinical electrographic seizure; and
generating a pro-ictal state alert corresponding to the pro-ictal state identified in the identifying step.

19. The method of claim 18 wherein the identifying step comprises identifying a pro-ictal state corresponding with a seizure onset characteristic and generating step comprises generating a pro-ictal state alert corresponding to the seizure onset characteristic.

20. The method of claim 19 wherein the seizure onset characteristic comprises a first seizure onset characteristic, identifying step further comprises identifying a pro-ictal state corresponding with a second seizure onset characteristic and the generating step comprises generating a pro-ictal state alert corresponding to the second seizure onset characteristic.

21. The method of claim 20 wherein the pro-ictal state alert corresponding to the first seizure onset characteristic is distinct from the pro-ictal state alert corresponding to the second seizure onset characteristic.

22. The method of claim 18 further comprising identifying a pro-ictal state correlated with a subclinical electrographic seizure; and generating a subclinical pro-ictal state alert corresponding to the pro-ictal state correlated with the subclinical electrographic seizure.

23. The method of claim 22 wherein the pro-ictal state alert corresponding to the pro-ictal state correlated with the clinical electrographic seizure is distinct from the subclinical pro-ictal state alert corresponding to the pro-ictal state correlated with the subclinical electrographic seizure.

24. The method of claim 20 further comprising generating no pro-ictal alerts correlated with subclinical electrographic seizures.

25. The method of claim 20 further comprising identifying within the EEG data a pro-ictal state correlated with a subclinical electrographic seizure and suppressing a pro-ictal state alert if a pro-ictal state is also correlated with a clinical electrographic seizure within the same EEG data.

26. The method of claim 20 further comprising identifying within the EEG data a pro-ictal state correlated with a subclinical electrographic seizure and generating a subclinical pro-ictal state alert distinct from the pro-ictal state alert if a pro-ictal state is also correlated with a clinical electrographic seizure within the same EEG data.

27. The method of claim 18 further comprising automatically providing a therapy to the subject in response to the alert.

28. The method of claim 18 further comprising automatically providing distinct therapies to the subject in response to alerts corresponding to distinct seizure onset characteristics.

29. The method of claim 18 further comprising automatically providing distinct therapies to the subject in response to alerts correlated with clinical and subclinical electrographic seizures.

30. A method of developing a seizure prediction system, comprising:
analyzing a patient EEG data set including clinical electrographic seizures and subclinical electrographic seizures; and
developing a seizure prediction algorithm for predicting seizures based on brain states preferentially correlated with clinical electrographic seizures over subclinical electrographic seizures.

31. The method of claim 30, further comprising:
storing the seizure prediction algorithm in memory of seizure prediction system.
